(19) United States
(12) Patent Application Publication
(10) Pub. No.: US 2022/0192638 A1
(43) Pub. Date: Jun. 23, 2022

(54) METHOD AND DEVICE FOR ANALYSIS OF ULTRASOUND IMAGE IN FIRST TRIMESTER OF PREGNANCY

(71) Applicants: UNIVERSITY OF ULSAN FOUNDATION FOR INDUSTRY COOPERATION, Ulsan (KR); THE ASAN FOUNDATION, Seoul (KR)

(72) Inventors: Chong Jai KIM, Seoul (KR); Eun Na KIM, Seoul (KR); Joong Yeup LEE, Seoul (KR); Do Yeong HWANG, Seoul (KR); Ki Chul KIM, Seoul (KR); Jinyoung MA, Seoul (KR)

(73) Assignees: UNIVERSITY OF ULSAN FOUNDATION FOR INDUSTRY COOPERATION, Ulsan (KR); THE ASAN FOUNDATION, Seoul (KR)

(21) Appl. No.: 17/602,611

(22) PCT Filed: Apr. 8, 2020
(86) PCT No.: PCT/KR2020/004780

§ 371 Date: Oct. 8, 2021

(30) Foreign Application Priority Data
Apr. 8, 2019 (KR) 10-2019-0040816

(51) Int. Cl. A61B 8/08; G06N 20/00; G16H 50/70

(52) U.S. Cl. CPC A61B 8/5223; A61B 8/0866; G16H 50/70; G06N 20/00; A61B 8/5207

(57) ABSTRACT

A method for analyzing an ultrasound image in the first trimester of pregnancy includes: acquiring an ultrasound image in the first trimester of pregnancy; and acquiring at least one of characteristics of uterus, fetus, placenta, gestational sac, and egg yolk related to the acquired ultrasound image, and determining a group pertinent to the acquired ultrasound image among a plurality of predesignated groups based on the acquired characteristic and the acquired ultrasound image, using an ultrasound image analysis device that has learned in a machine learning technique.

### TECHNICAL FIELD

The present disclosure relates to a method and device for analyzing an ultrasound image acquired in the first trimester of pregnancy.

### BACKGROUND ART

Recently, as the age of first marriage increases, the number of high-risk childbirths caused by the pregnancy at an advanced maternal age is increasing. According to statistical figures, the age of first marriage for women was 25.1 in 1994, whereas the age of first marriage for women increased to 30.0 years in 2015,and the number of pregnant women over 35 also increased. For aged pregnant women, e.g. pregnant women over 40, a miscarriage rate is roughly 50%, so that there is a great risk of high-risk childbirth.

For high-risk pregnant women, it is necessary to periodically and continuously manage a pregnant state. However, devices for general patients, for example, devices such as magnetic resonance imaging devices, may press down on the inferior vena cava of pregnant women or may expose the fetus to a magnetic field, thus limiting the use of the devices. Thus, an ultrasound device is widely used to track and manage the course of a pregnancy while ensuring the safety of a pregnant woman and a fetus.

Meanwhile, an ultrasound image acquired by the ultrasound device is problematic in that it is difficult for an unskilled obstetrician to diagnose the risk of miscarriage, so the ultrasound image is not actively used to determine the course of pregnancy of high-risk pregnant women. Accordingly, there is a need for technology capable of using the ultrasound image to determine the course of pregnancy even by an unskilled obstetrician.

### DOCUMENTS OF RELATED ART

Korean Patent No. 10-1097645 registered on Dec. 15, 2011)

### DETAILED DESCRIPTION OF THE DISCLOSURE

### Technical Problem

The present disclosure provides more precise information about a pregnant state by analyzing an ultrasound image in the first trimester of pregnancy.

Technical objects to be achieved by the present disclosure are not limited to the aforementioned technical objects, and other technical objects not described above may be evidently understood by a person having ordinary skill in the art to which the present disclosure pertains from the following description.

### Technical Solution to the Problem

In accordance with an aspect of the present disclosure, there is provided a method for analyzing an ultrasound image in the first trimester of pregnancy, the method including: acquiring an ultrasound image in the first trimester of pregnancy; and acquiring at least one of characteristics of uterus, fetus, placenta, gestational sac, and egg yolk related to the acquired ultrasound image, and determining a group pertinent to the acquired ultrasound image among a plurality of predesignated groups based on the acquired characteristic and the acquired ultrasound image, using an ultrasound image analysis device that has learned in a machine learning technique.

The characteristics of the uterus may include a texture of the uterus, a density of the uterus, and a shape of the uterus. The characteristics of the fetus may include a texture of the fetus, a density of the fetus, a size of the fetus, and a shape of the fetus. The characteristics of the placenta may include a texture of the placenta, a density of the placenta, a size of the placenta, a shape of the placenta, and a change in a cyst in the placenta. The characteristics of the gestational sac may include a number of gestational sacs, a texture of the gestational sac, a density of the gestational sac, a size of the gestational sac, and a shape of the gestational sac. The characteristics of the egg yolk may include a texture of the egg yolk, a density of the egg yolk, a size of the egg yolk, and a shape of the egg yolk.

The ultrasound image analysis device may designate a plurality of ultrasound images in the first trimester of pregnancy, which are pre-stored in a learning database, such that each of the plurality of ultrasound images in the first trimester of pregnancy is included in at least one of the plurality of predesignated groups, based on the at least one characteristic.

Learning the ultrasound image analysis device in the machine learning technique may include: clustering the plurality of ultrasound images in the first trimester of pregnancy, which are pre-stored in the learning database, in a plurality of groups based on the at least one characteristic, in a process of learning the ultrasound image analysis device in the machine learning technique; and connecting the clustered groups to the plurality of predesignated groups, respectively.

The plurality of predesignated groups may include at least two of a multifetal group, a molar pregnancy group, a fetal genetic risk group, a fetal growth restriction group, a miscarriage risk group, a decidual abnormality group, a villous abnormality group, and a normal group.

The ultrasound image and at least one of the characteristics of the uterus, the fetus, the placenta, the gestational sac, and the egg yolk, which are related to the acquired ultrasound image, may be received from an external ultrasound acquisition device.

The ultrasound image may be received from the external ultrasound acquisition device, and at least one of the characteristics of the uterus, the fetus, the placenta, the gestational sac, and the egg yolk, which are related to the acquired ultrasound image, may be extracted from the ultrasound image by the ultrasound image analysis device.

In accordance with another aspect of the present disclosure, there is provided a device for analyzing an ultrasound image in the first trimester of pregnancy, the device including: an image acquisition unit configured to acquire the ultrasound image in the first trimester of pregnancy; and a group determination unit configured to acquire at least one of the characteristics of the uterus, the fetus, the placenta, the gestational sac, and the egg yolk, which are related to the acquired ultrasound image, to perform learning in a machine learning technique on the basis of the acquired characteristics and the acquired ultrasound image, and to determine a group pertinent to the acquired ultrasound image among a plurality of predesignated groups based on the learning.

### Advantageous Effects of the Disclosure

A method and device for analyzing an ultrasound image in the first trimester of pregnancy according to an embodiment of the present disclosure provide more precise information about a pregnant state by analyzing an ultrasound image in the first trimester of pregnancy, thus allowing the risk of pregnancy to be more precisely tracked and managed in the first trimester of pregnancy, i.e., an early stage of pregnancy.

Effects of the present disclosure are not limited to the aforementioned effects, and other effects not described above may be evidently understood by a person having ordinary skill in the art to which the present disclosure pertains from the claims.

### BRIEF DESCRIPTION OF THE DRAWING

FIGS. 1A and 1B conceptually illustrate an example in which an ultrasound image in the first trimester of pregnancy is acquired and the acquired ultrasound image, according to an embodiment of the present disclosure.

FIGS. 2A and 2B illustrate an example of the ultrasound image in the first trimester of pregnancy, according to the embodiment of the present disclosure.

FIG. 3 conceptually illustrates a method for analyzing an ultrasound image in the first trimester of pregnancy, according to the embodiment of the present disclosure.

FIG. 4 is a functional block diagram illustrating a device for analyzing an ultrasound image in the first trimester of pregnancy, according to the embodiment of the present disclosure.

FIG. 5 is a flowchart illustrating a method for analyzing an ultrasound image in the first trimester of pregnancy, according to the embodiment of the present disclosure.

### MODE FOR CARRYING OUT THE DISCLOSURE

Advantages and features of the present disclosure, and a method of achieving them will become apparent with reference to an embodiment described later together with the accompanying drawings. However, the present disclosure is not limited to an embodiment disclosed below, but may be implemented in a variety of different forms. That is, the embodiment is provided to ensure that descriptions of the present disclosure are complete and to fully inform a scope of the disclosure to a person with ordinary knowledge in a technical field to which the present disclosure belongs, and the disclosure is only defined by the scope of claims.

In describing the embodiments of the present disclosure, the detailed descriptions of well-known functions or configurations will be omitted if it is determined that the detailed descriptions of well-known functions or configurations may unnecessarily make obscure the spirit of the present disclosure. The terms used henceforth are defined in consideration of the functions of the disclosure and may be altered according to the intent of a user or operator, or conventional practice. Therefore, the terms should be defined on the basis of the entire content of this disclosure.

The present disclosure may make various changes and include various embodiments, and specific embodiments will be illustrated in the drawings and described in the detailed description. However, the present disclosure is not limited to a specific embodiment, and it should be understood that the present disclosure includes all changes, equivalents, or substitutes included in the spirit and scope thereof.

Terms including ordinal numbers, such as first and second, may be used for describing various elements, but the corresponding elements are not limited by these terms. These terms are only used for the purpose of distinguishing one element from another element.

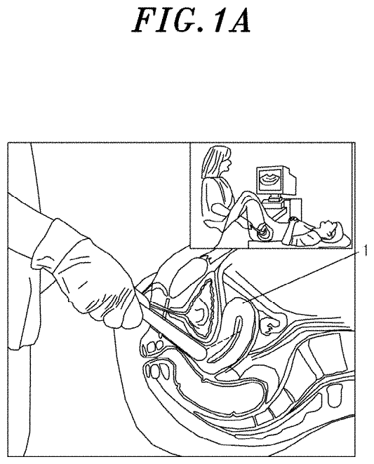

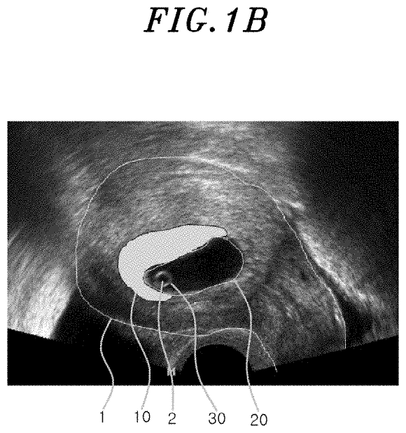

FIGS. 1A and 1B conceptually illustrate an embodiment in which an ultrasound image in the first trimester of pregnancy is acquired and the acquired ultrasound image, according to an embodiment of the present disclosure. More specifically, FIG. 1A illustrates a method for analyzing the ultrasound image in the first trimester of pregnancy, and FIG. 1B illustrates the ultrasound image in the first trimester of pregnancy, which is acquired according to the embodiment.

Referring to FIG. 1A, an ultrasound device may be inserted into the body of a pregnant woman to acquire an ultrasound image of a component such as an uterus, a fetus in the uterus, a placenta, a gestational sac, or an egg yolk. The ultrasound device may acquire information about the fetus and the uterus of the pregnant woman in a non-invasive manner, and may be used to diagnose a pregnant state in the first trimester of pregnancy.

Hereinafter, the ultrasound image of this specification may refer to the ultrasound image of the first trimester of pregnancy, which is acquired from the pregnant woman in the first trimester of pregnancy.

FIG. 1B shows the ultrasound image acquired by the method of FIG. 1A. As shown in FIG. 1B, the ultrasound image may include a uterus 1, and the uterus 1 may include a fetus 2, a placenta 10, a gestational sac 20, and an egg yolk 30. Thus, the characteristics of the fetus 2, the placenta 10, the gestational sac 20, and the egg yolk 30 may be distinguished using the ultrasound image.

More specifically, the fetus 2, the placenta 10, the gestational sac 20, and the egg yolk 30 may be included in the uterus 1. The fetus 2 may be a part of the egg yolk 30, for example, a part that appears as if a part of the ring-shaped egg yolk is deleted, or a part corresponding to a diamond attached to a ring assuming that the egg yolk has a ring shape. The placenta 10 and the gestational sac 20 may be adjacent to each other, and the ring-shaped egg yolk 30 may be included in the gestational sac 20.

As described above, the ultrasound image may include the images of the fetus 2, the placenta 10, the gestational sac 20, and the egg yolk 30. The characteristics of each component may be changed depending on the course of pregnancy or the risk degree of miscarriage.

Meanwhile, the characteristics of the fetus 2 may include, for example, the texture of the fetus 2, the density of the fetus 2, the size of the fetus 2, and the shape of the fetus 2, the characteristics of the placenta 10 may include, for example, the texture of the placenta 10, the density of the placenta 10, the size of the placenta 10, the shape of the placenta 10, and a change in a cyst in the placenta 10, and the characteristics of the gestational sac 20 may include, for example, the number of gestational sacs 20, the texture of the gestational sac 20, the density of the gestational sac 20, the size of the gestational sac 20, and the shape of the gestational sac 20. The characteristics of the egg yolk 30 may include the texture of the egg yolk 30, the density of the egg yolk 30, the size of the egg yolk 30, and the shape of the egg yolk 30.

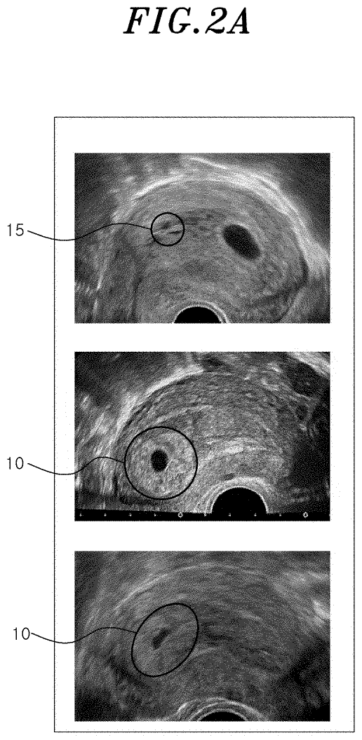

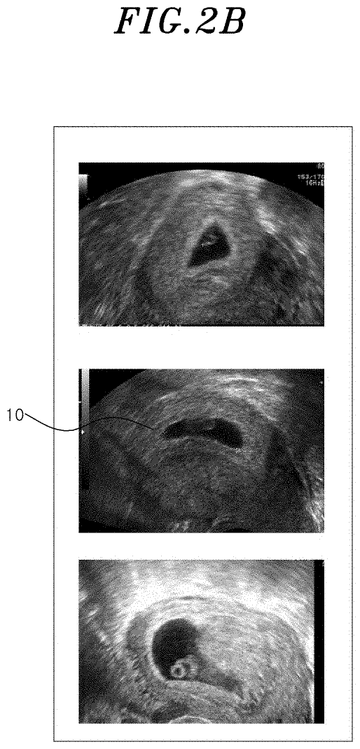

FIGS. 2A and 2B illustrate an example of the ultrasound image in the first trimester of pregnancy, according to the embodiment of the present disclosure. To be more specific, FIGS. 2A and 2B illustrate an example of an ultrasound image showing characteristics in which the risk of miscarriage is present and an example of an ultrasound image showing normal characteristics.

FIG. 2A shows the ultrasound image of a pregnant woman group having characteristics in which the risk of miscarriage is present, and FIG. 2B shows the ultrasound image of a pregnant woman group in a normal condition having no risk of miscarriage.

Referring to FIG. 2A, the ultrasound image of the pregnant woman group having the risk of miscarriage may vary depending on the cause of the risk of miscarriage. For example, when a villus proliferates due to molar pregnancy to have the risk of miscarriage, the density of the placenta 10 may be increased to a predetermined value or more. Further, in the case of having the risk of miscarriage due to the villus abnormality, the placenta 10 may be shaped to have a predetermined thickness or more.

In some cases, when there is a risk of miscarriage due to an abnormality in the decidua, as shown in the first ultrasound image of FIG. 2A, a hole 15 may be included in the uterus 1 in the ultrasound image.

Meanwhile, referring to FIG. 2B, the placenta 10 in the ultrasound image of the pregnant woman group having no risk of miscarriage may have a thickness less than a predetermined value, and the shape of the placenta may be evenly formed.

Depending on the specific cause of the miscarriage, the characteristics of components (e.g. uterus 1, placenta 10, gestational sac 20, and egg yolk 30) included in the ultrasound image may be different.

As described above, the characteristics of the component included in the ultrasound image of the pregnant woman group having the possibility of the miscarriage (or the probability of the miscarriage is equal to or more than a predetermined value) may be different from the characteristics of the component included in the ultrasound image of the pregnant woman group having no risk of the miscarriage (or the probability of the miscarriage is less than the predetermined value). Further, the causes according to these characteristics may also be different from each other.

Meanwhile, the characteristics of the component included in the ultrasound image depending on whether there is a risk of miscarriage with reference to FIGS. 2A and 2B are not limited to the above description.

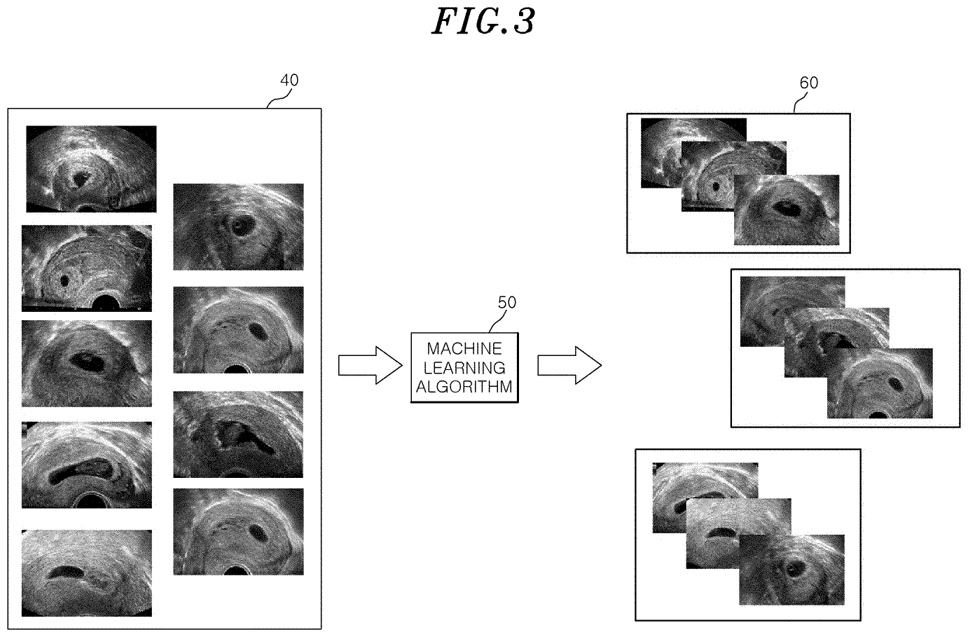

FIG. 3 conceptually illustrates a method for analyzing an ultrasound image in the first trimester of pregnancy, according to the embodiment of the present disclosure. To be more specific, FIG. 3 shows the analysis method using a machine learning algorithm after acquiring the ultrasound image in the first trimester of pregnancy.

Referring to FIG. 3, a plurality of ultrasound images 40 may be acquired for the first trimester of pregnancy. The plurality of ultrasound images 40 may be acquired and transmitted by an external ultrasound device, or an ultrasound image analysis device may include the ultrasound device to directly acquire the ultrasound images. The acquisition method is not limited thereto.

Further, at least one of the characteristics of the uterus, fetus, placenta, gestational sac, and egg yolk related to the acquired ultrasound image may be received from an external ultrasound acquisition device.

Further, the ultrasound image may be received from the external ultrasound acquisition device, and at least one of the characteristics of the uterus, fetus, placenta, gestational sac, and egg yolk related to the acquired ultrasound image may be extracted from the acquired ultrasound image.

The plurality of ultrasound images 40 may include an ultrasound image of a pregnant woman group having the risk of miscarriage. In some cases, the plurality of ultrasound images 40 may further include an ultrasound image of a normal pregnant woman group.

As described above with reference to FIGS. 1A and 1B or FIGS. 2A and 2B, the ultrasound image 40 may show at least one of the characteristics of the uterus 1, fetus 2, placenta 10, gestational sac 20, and egg yolk 30. These characteristics may characteristics showing or related to the risk of the miscarriage.

For example, the thickness of the placenta 10 of the ultrasound image having the risk of miscarriage may be equal to or more than a predetermined value. Meanwhile, when the ultrasound image of the pregnant woman group having no risk of miscarriage is included in the ultrasound image 40, the thickness of the placenta 10 shown in the ultrasound image may be less than a predetermined value.

The plurality of ultrasound images 40 may be analyzed by the machine learning algorithm 50 to be clustered according to each characteristic.

The machine learning algorithm 50 may be a pre-learned algorithm to classify the ultrasound images according to the characteristics using various ultrasound images. For example, the characteristics classifying the ultrasound images may include the ultrasound texture of the uterus, the density of the uterus, the shape of the uterus, the ultrasound texture of the fetus, the density of the fetus, the size of the fetus, the shape of the fetus, the ultrasound texture of the placenta, the density of the placenta, the size of the placenta, the shape of the placenta, a change in a cyst in the placenta, the number of gestational sacs, the ultrasound texture of the gestational sac, the density of the gestational sac, the size of the gestational sac, the shape of the gestational sac, the ultrasound texture of the egg yolk, the density of the egg yolk, the size of the egg yolk, and the shape of the egg yolk.

The machine learning algorithm 50 may be learned by supervised learning or unsupervised learning. For example, the machine learning algorithm 50 may be learned by designating the plurality of ultrasound images pre-stored in a database such that each of the plurality of ultrasound images is included in at least one of predesignated groups according to at least one characteristic.

In another example, the machine learning algorithm 50 may be learned such that the plurality of pre-stored ultrasound images is clustered according to at least one characteristic, with respect to the plurality of ultrasound images pre-stored in the database.

In some cases, the machine learning algorithm 50 may be learned to more delicately classify the clustering of the ultrasound image using various pre-stored pieces of information as well as the ultrasound image. Various pre-stored pieces of information may be information about a pregnant woman (or fetus) according to the ultrasound image, and may include, for example, the age of the pregnant woman, a last menstrual period (LMP), and a human chorionic gonadotropin level.

By clustering the ultrasound images by the machine learning algorithm 50, the plurality of ultrasound images 40 may be divided into at least two groups. At least two groups may include a first group 60, a second group 70, and a third group 80.

At least two groups may include groups divided according to the state of a fetus or the state of pregnancy. For example, the first group 60 may be a group of an ultrasound image of a normal state, the second group 70 may be a fetal genetic risk group, and the third group 80 may be a fetal growth restriction group.

In some cases, each of the groups divided according to characteristics may be classified according to the specific cause of miscarriage. For example, the first group 60 may be a molar pregnancy group, the second group 70 may be a decidual abnormality group, and the third group 80 may be a villous abnormality group.

However, the specific example of at least one group is not limited to the above-described example, and may include a group showing various states divided on the basis of at least one of the characteristics of the uterus 1, the fetus 2, the placenta 10, the gestational sac 20, and the egg yolk 30.

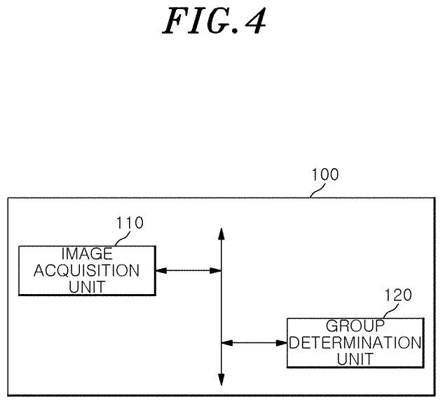

FIG. 4 is a functional block diagram illustrating a device for analyzing an ultrasound image in the first trimester of pregnancy, according to the embodiment of the present disclosure. The term “unit” used hereinafter may mean a unit processing at least one function or operation, which may be implemented as hardware, software, or a combination of hardware and software.

Referring to FIG. 4, the ultrasound image analysis device 100 may include an image acquisition unit 110 and a group determination unit 120. The image acquisition unit 110 may be implemented by a calculation device including a microprocessor, and the same also applies to the parameter group determination unit 120 that will be described later.

The image acquisition unit 110 may acquire the ultrasound image in the first trimester of pregnancy. The image acquisition unit 110 may directly acquire the ultrasound image, and may receive and acquire the ultrasound image from another device.

Further, the ultrasound image analysis device 100 may receive at least one of characteristics of the uterus, fetus, placenta, gestational sac, and egg yolk, related to the ultrasound image acquired by the image acquisition unit 110, from the external ultrasound acquisition device.

Furthermore, the ultrasound image analysis device 100 may receive the ultrasound image from the external ultrasound acquisition device, and may extract at least one of characteristics of the uterus, fetus, placenta, gestational sac, and egg yolk, related to the acquired ultrasound image, from the acquired ultrasound image.

The group determination unit 120 may determine a group pertinent to the ultrasound image among a plurality of predesignated groups. More specifically, the group determination unit 120 may determine a group pertinent to the ultrasound image, using the machine learning algorithm 50 learned on the basis of at least one of the characteristics of the uterus 1, fetus 2, placenta 10, gestational sac 20, and egg yolk 30.

The machine learning algorithm 50 may be learned to cluster the ultrasound images according to the characteristics, using a plurality of pre-stored ultrasound images. For example, the machine learning algorithm 50 may be learned by designating the plurality of ultrasound images pre-stored in the database such that each of the plurality of ultrasound images in the first trimester of pregnancy is included in at least one of the predesignated groups according to at least one characteristic.

Further, the machine learning algorithm 50 may be learned such that the plurality of pre-stored ultrasound images is clustered according to at least one characteristic, with respect to the plurality of ultrasound images pre-stored in the database. Thus, when a new ultrasound image is acquired and input into the machine learning algorithm 50, the ultrasound image may be classified into any one of the groups generated by clustering. However, in some cases, the acquired ultrasound image may naturally belong to two or more groups among a plurality of predesignated groups.

Meanwhile, the plurality of predesignated groups may be groups generated by the clustering of the machine learning algorithm 50. For example, the plurality of predesignated groups may include at least two of a multifetal group, a molar pregnancy group, a fetal genetic risk group, a fetal growth restriction group, a miscarriage risk group, a decidual abnormality group, and a villous abnormality group.

Further, in some cases, the group determination unit 120 may analyze similarity for each group of the ultrasound image to provide information about the similarity. The information about the similarity may be provided in various ways. For example, the information about the similarity may be provided in the form of a graph and a number. Further, the machine learning algorithm 50 may be learned to estimate similarity related to the plurality of predesignated groups, with respect to the ultrasound image, on the basis of the characteristics of the uterus 1, fetus 2, placenta 10, gestational sac 20, and egg yolk 30.

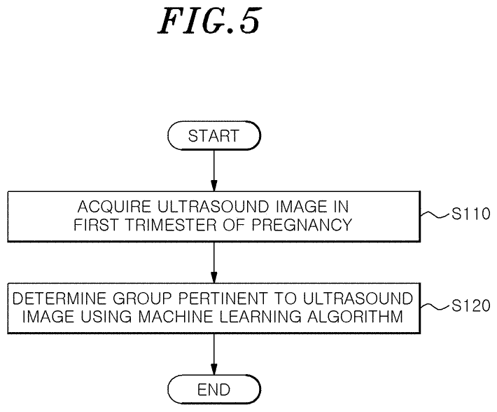

FIG. 5 is a flowchart illustrating respective steps of a method for analyzing an ultrasound image in the first trimester of pregnancy, according to the embodiment of the present disclosure. Of course, the respective steps of the method shown in FIG. 5 may be performed in an order different from that illustrated in the drawing if necessary. Hereinafter, the duplicated description of FIGS. 4 and 5 will be omitted.

Referring to FIG. 5, the image acquisition unit 110 may acquire the ultrasound image in the first trimester of pregnancy (step S110). The uterus 1, fetus 2, placenta 10, gestational sac 20, and egg yolk 30 may be shown in the ultrasound image in the first trimester of pregnancy. Their characteristics may be differently shown depending on the state of pregnancy, for example, depending on whether there is a risk of miscarriage.

The group determination unit 120 may determine a group pertinent to the ultrasound image, using the machine learning algorithm 50 (step S120).

The machine learning algorithm 50 may be learned by designating the plurality of ultrasound images in the first trimester of pregnancy, which are pre-stored in the database, such that each of the plurality of ultrasound images in the first trimester of pregnancy is included in at least one of the predesignated groups according to the characteristics of any one of the uterus 1, fetus 2, placenta 10, gestational sac 20, and egg yolk 30 included in the ultrasonic image. Thus, the group determination unit 120 may determine such that the ultrasound image acquired by the image acquisition unit 110 belongs to at least one of the plurality of predesignated groups, using the machine learning algorithm 50.

The machine learning algorithm 50 may be learned to cluster the plurality of pre-stored ultrasound images in the first trimester of pregnancy on the basis of the characteristics of any one of the uterus 1, fetus 2, placenta 10, gestational sac 20, and egg yolk 30 included in the ultrasonic image. In this case, the group determination unit 120 may connect each clustered group to the predesignated group, and may use the machine learning algorithm 50 to determine that the ultrasound image belongs to at least one of the groups generated by clustering.

The ultrasound image analysis device 100 according to the embodiment of the present disclosure can diagnose the pregnant state from the first trimester of pregnancy, by analyzing the ultrasound image in the first trimester of pregnancy, which is difficult to track the pregnant state, on the basis of at least one of the characteristics of the uterus, fetus, placenta, gestational sac, and egg yolk.

The ultrasound image analysis device 100 according to the embodiment of the present disclosure can more rapidly and precisely provide information about the pregnant state, by automatically analyzing the ultrasound image in the first trimester of pregnancy using the machine learning algorithm.

Combinations of each block in the block diagram and each step in flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each block of the block diagram or each step of the flowchart. The computer program instructions can also be stored on a computer-usable or computer-readable recording medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each block of the block diagram or each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each block of the block diagram or each step of the flowchart.

In addition, each block or each step may represent a module, a segment, or a portion of codes which contains one or more executable instructions for executing the specified logical function(s). It should also be noted that in some alternative embodiments, the functions mentioned in the blocks or the steps may occur out of order. For example, two blocks or steps illustrated in succession may in fact be performed substantially simultaneously, or the blocks or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

1. A method for analyzing an ultrasound image in the first trimester of pregnancy, the method comprising:
acquiring an ultrasound image in the first trimester of pregnancy; and
acquiring at least one of characteristics of uterus, fetus, placenta, gestational sac, and egg yolk related to the acquired ultrasound image, and determining a group pertinent to the acquired ultrasound image among a plurality of predesignated groups based on the acquired characteristic and the acquired ultrasound image, using an ultrasound image analysis device that has learned in a machine learning technique.

2. The method of claim 1, wherein the characteristics of the uterus include a texture of the uterus, a density of the uterus, and a shape of the uterus,
the characteristics of the fetus include a texture of the fetus, a density of the fetus, a size of the fetus, and a shape of the fetus,
the characteristics of the placenta include a texture of the placenta, a density of the placenta, a size of the placenta, a shape of the placenta, and a change in a cyst in the placenta,
the characteristics of the gestational sac include a number of gestational sacs, a texture of the gestational sac, a density of the gestational sac, a size of the gestational sac, and a shape of the gestational sac, and
the characteristics of the egg yolk include a texture of the egg yolk, a density of the egg yolk, a size of the egg yolk, and a shape of the egg yolk.

3. The method of claim 1, wherein the ultrasound image analysis device designates a plurality of ultrasound images in the first trimester of pregnancy, which are pre-stored in a learning database, such that each of the plurality of ultrasound images in the first trimester of pregnancy is included in at least one of the plurality of predesignated groups, based on the at least one characteristic.

4. The method of claim 3, wherein learning the ultrasound image analysis device in the machine learning technique comprises:
clustering the plurality of ultrasound images in the first trimester of pregnancy, which are pre-stored in the learning database, in a plurality of groups based on the at least one characteristic, in a process of learning the ultrasound image analysis device in the machine learning technique; and
connecting the clustered groups to the plurality of predesignated groups, respectively.

5. The method of claim 1, wherein the plurality of predesignated groups includes at least two of a multifetal group, a molar pregnancy group, a fetal genetic risk group, a fetal growth restriction group, a miscarriage risk group, a decidual abnormality group, a villous abnormality group, and a normal group.

6. The method of claim 1, wherein the ultrasound image and at least one of the characteristics of the uterus, the fetus, the placenta, the gestational sac, and the egg yolk, which are related to the acquired ultrasound image, are received from an external ultrasound acquisition device.

7. The method of claim 1, wherein the ultrasound image is received from an external ultrasound acquisition device, and
at least one of the characteristics of the uterus, the fetus, the placenta, the gestational sac, and the egg yolk, which are related to the acquired ultrasound image, is extracted from the ultrasound image by the ultrasound image analysis device.

8. A device for analyzing an ultrasound image in the first trimester of pregnancy, the device comprising:
an image acquisition unit configured to acquire the ultrasound image in the first trimester of pregnancy; and
a group determination unit configured to acquire at least one of the characteristics of the uterus, the fetus, the placenta, the gestational sac, and the egg yolk, which are related to the acquired ultrasound image, to perform learning in a machine learning technique on the basis of the acquired characteristics and the acquired ultrasound image, and to determine a group pertinent to the acquired ultrasound image among a plurality of predesignated groups based on the learning.

9. The method of claim 2, wherein the plurality of predesignated groups includes at least two of a multifetal group, a molar pregnancy group, a fetal genetic risk group, a fetal growth restriction group, a miscarriage risk group, a decidual abnormality group, a villous abnormality group, and a normal group.

10. The method of claim 3, wherein the plurality of predesignated groups includes at least two of a multifetal group, a molar pregnancy group, a fetal genetic risk group, a fetal growth restriction group, a miscarriage risk group, a decidual abnormality group, a villous abnormality group, and a normal group.

11. The method of claim 4, wherein the plurality of predesignated groups includes at least two of a multifetal group, a molar pregnancy group, a fetal genetic risk group, a fetal growth restriction group, a miscarriage risk group, a decidual abnormality group, a villous abnormality group, and a normal group.
